(19) United States
(12) Patent Application Publication
(10) Pub. No.: US 2018/0263810 A1
(43) Pub. Date: Sep. 20, 2018

(54) Male Condom Assembly with Integral Trapezoidal-shaped Shield for Inhibiting Skin Contact along The Genitals and Testicles

(71) Applicant: Marcos Eduardo Lopez, Los Angeles, CA (US)

(72) Inventor: Marcos Eduardo Lopez, Los Angeles, CA (US)

(21) Appl. No.: 15/925,679

(22) Filed: Mar. 19, 2018

Related U.S. Application Data
(60) Provisional application No. 62/473,380, filed on Mar. 19, 2017.

(51) Int. Cl. A61F 6/04

(52) U.S. Cl. CPC A61F 6/04; A61F 2006/041

(57) ABSTRACT

A male condom assembly with trapezoidal-shaped shield for inhibiting skin contact along the genitals and testicles. The male condom assembly provides a flexible, thin-walled tube that positions over male genitals, and specifically the penis. The tube has a closed distal end and an open base end. The open base end of the tube is defined by a reinforced perimeter region that secures the base end of the tube to the base of the penis. A trapezoidal-shaped shield is integrally connected with at least half the circumference of the reinforced perimeter of the tube, at the base end thereof. The shield orients downwardly to align with the testicles during intercourse. The shield may be fabricated from an adhesive material that helps the shield wrap around the testicles, and at least partially cover the scrotum, pubic hair, and peripheral area of genitals.

### CROSS REFERENCE OF RELATED APPLICATIONS

This application claims the benefits of U.S. provisional application No. 62/473,380, filed Mar. 19, 2017 and entitled THE EMBODIMENT IS A CONDOM WITH A TRAPEZOID LIKE PAD ATTACHED TO THE BOTTOM, which provisional application is incorporated by reference herein in its entirety.

### FIELD OF THE INVENTION

The present invention relates generally to a male condom assembly with trapezoidal-shaped shield for inhibiting skin contact along the genitals and testicles. More so, the present invention relates to a male condom assembly that provides full coverage around the genital area and along the length of the testicles during intercourse, so as to effectively inhibit sexually transmitted diseases and pregnancy, and to at least partially wrap around the testicles so that the condom does not fall off when the penis softens after intercourse; whereby the condom assembly comprises a flexible, thin-walled tube having a closed distal end and an open base end defined by a reinforced perimeter to position over male genitalia; and a trapezoidal-shaped shield that is integrally connected with at least half the circumference of the reinforced perimeter of the tube, and further the shield that sticks to the testicles during intercourse.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, a male standard condom is an elongated cylindrical sheath of thin latex or plastic worn on the penis during intercourse. The male condom is designed to capture semen before, during, and after a man ejaculates. This can keep sperm from a male to a female vagina to prevent pregnancy and some sexual transmitted diseases.

Generally, the standard sheath-like condom only covers the penis of the male. It cannot provide an impermeable barrier for areas of the skin other than the shaft of the penis that come into contact during sexual intercourse. However, some STDs can be transmitted through skin-to-skin contact (specifically skin with damage) such as human papillomavirus (HPV), and condoms offer little protection against these STDs. Pubic lice can be transmitted by direct physical contact with pubic hair. Another disadvantage of the standard condom is that the man must withdraw immediately after ejaculation so that the condom does not fall off when the penis softens.

Other proposals have involved condoms that cover the genital area. The problem with these condoms is that they do not have a shield that is sized and dimensioned to extend down along the length of the testicles. Also, any barrier that has been used does not adhere to the testicles, or pubic. Even though the above cited condoms meet some of the needs of the market, a male condom assembly with trapezoidal-shaped shield that inhibits skin contact along the genitals and testicles by providing full coverage around the genital area and along the length of the testicles during intercourse, is still desired.

### SUMMARY

Illustrative embodiments of the disclosure are generally directed to a male condom assembly with trapezoidal-shaped shield for inhibiting skin contact along the genitals and testicles. The male condom assembly serves to provide full coverage around the genital area and along the length of the testicles during intercourse, so as to effectively inhibit sexually transmitted diseases and pregnancy, and to at least partially wrap around the testicles; so that the genitals and testicles are inhibited from skin contact, and also so that the male condom assembly does not fall off when the penis softens after intercourse.

In some embodiments, the condom assembly comprises a flexible, thin-walled tube to position over male genitals, and specifically the penis. The tube has a closed distal end and an open base end. The open base end of the tube is defined by a reinforced perimeter region that secures the base end of the tube to the base of the penis.

In some embodiments, the condom assembly further comprises a trapezoidal-shaped shield that is integrally connected with at least half the circumference of the reinforced perimeter of the tube, at the base end thereof. The shield is configured to orient in alignment with the testicles during intercourse. The shield may be fabricated from an adhesive material that helps the shield cling, and thereby wrap around the testicles, and at least partially cover the scrotum, pubic hair, and peripheral area of the genitals.

One aspect of a male condom assembly with trapezoidal-shaped shield, comprises:
a flexible, thin-walled tube comprising by a closed distal end and an open base end, the open base end comprising a perimeter region defined by a circumference; and
a trapezoidal-shaped shield comprising an attached end and a free end, the attached end of the shield being integrally connected with at least half the circumference of the reinforced perimeter of the tube,
whereby the free end of the shield aligns with and sticks to the testicles during intercourse.

In another aspect, the shield covers a front region and a side region of the testicles.

In another aspect, the shield comprises an adhesive material that helps the shield cling, and thereby adhere to the testicles.

In another aspect, the trapezoidal shape of the shield is generally rectangular.

In another aspect, the trapezoidal shape of the shield includes at least one of the following trapezoidal shapes: a right trapezoid, an acute trapezoid, an isosceles trapezoid, a parallelogram, a tangential trapezoid, a Saccheri quadrilateral, a rhombus, and a square.

In yet another aspect, the attached end of the shield has a generally shorter length than the free end of the shield.

In yet another aspect, the attached end of the shield forms a pair of obtuse angles.

In yet another aspect, the size of the shield is about 25×15 centimeters. Though in other embodiments, the shield can be customized to any size and dimension.

In yet another aspect, the thickness of the shield is from 0.01 millimeters to 0.25 millimeters.

In yet another aspect, the reinforced perimeter region is reinforced with latex, polyurethane, or plastic.

In yet another aspect, the perimeter region is defined by a generally circular shape.

In another aspect, the closed distal end of the tube has a generally smaller diameter than the open base end of the tube.

In another aspect, the closed distal end of the tube has a reservoir tip for containing semen.

In another aspect, the open base end forms a hole for enabling passage of the penis.

In yet another aspect, the tube comprises a material including at least one of the following: latex, polyurethane, animal skin, and a thin plastic material.

In yet another aspect, the thickness of the tube is from 0.01 millimeters to 0.25 millimeters.

In yet another aspect, the tube is sized and dimensioned to substantially cover a penis.

In yet another aspect, the tube is defined by an inner surface and an outer surface.

In yet another aspect, the tube is lubricated from the inner surface, the outer surface, or both to help enhance comfort during intercourse.

One objective of the present invention is to at least partially cover the genital area and testicles to inhibit skin contact during intercourse.

Another objective is to provide a shield having a generally trapezoidal shape that substantially forms a barrier between the testicles of a male and the skin of a female.

Yet another objective is to enable the shield to adhere to the testicles, so as to at least partially wrap around the testicles.

Yet another objective is to inhibit sexually transmitted diseases and pregnancy

Yet another objective is to fabricate the shield from an adhesive material that helps the shield cling, and thereby wrap around the testicles.

Yet another objective is to provide an inexpensive to manufacture male condom.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a front perspective view of an exemplary male condom assembly with trapezoidal-shaped shield, in accordance with an embodiment of the present invention; and

FIG. 2 illustrates a rear perspective view of the male condom assembly shown in FIG. 1, showing the thin-walled tube receiving a penis and the trapezoidal-shaped shield aligned with the testicles, in accordance with an embodiment of the present invention.

Like reference numerals refer to like parts throughout the various views of the drawings.

### DETAILED DESCRIPTION OF THE INVENTION

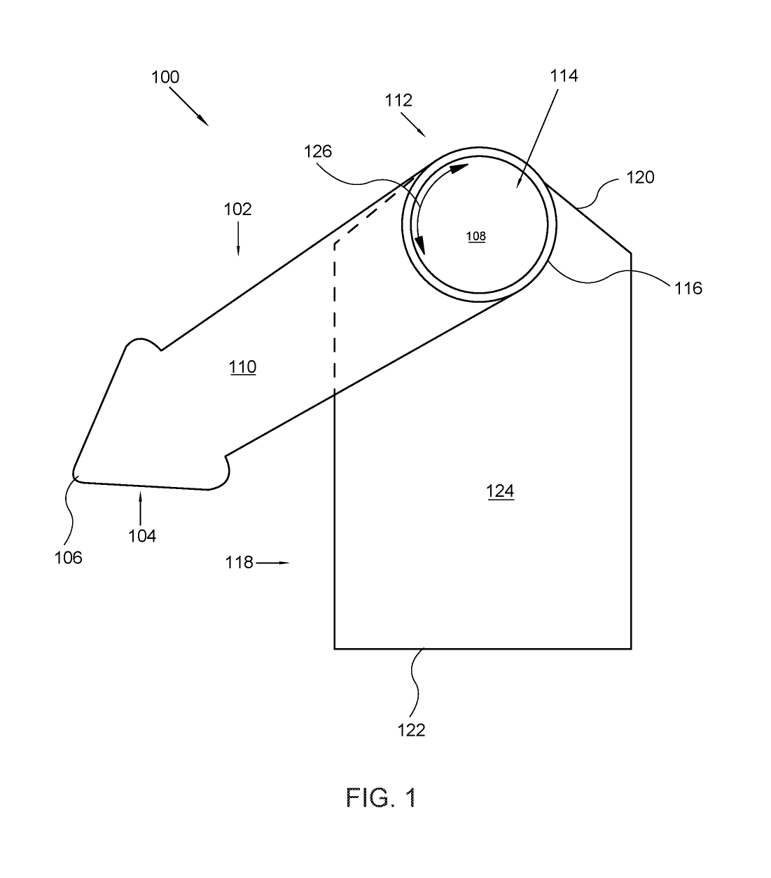

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

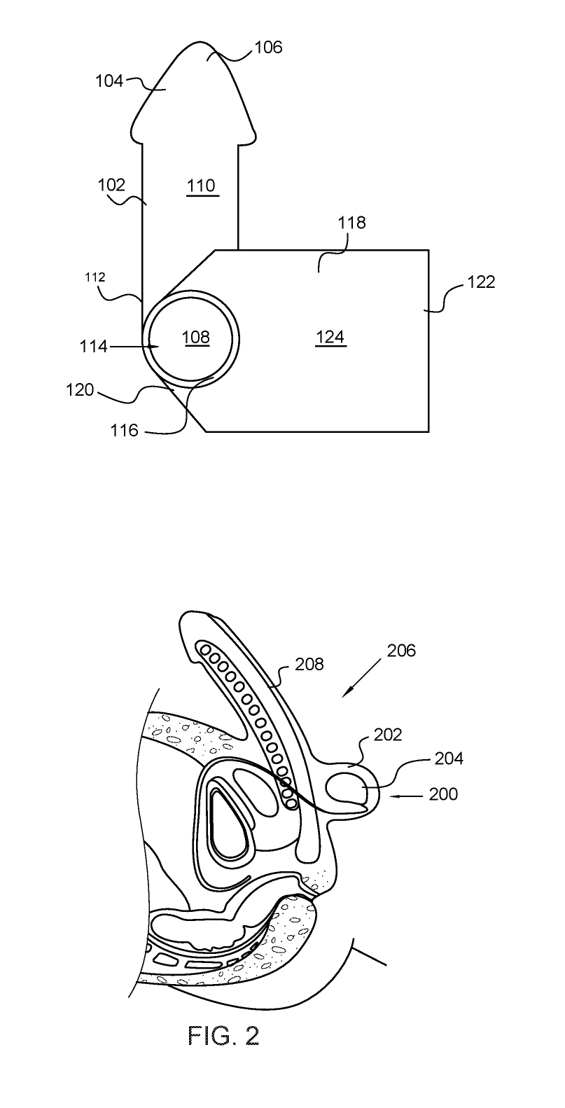

A male condom assembly 100 with trapezoidal-shaped shield for inhibiting skin contact along the genitals and testicles is referenced in FIGS. 1 and 2. The male condom assembly 100, hereafter “assembly 100” is configured to cover the genital 206 area, especially the penis 208 with a flexible thin-walled tube 102; and to at least partially wrap around the testicles 200, and at least partially cover the scrotum, pubic hair, and peripheral area of the genitals with a trapezoidal-shaped shield 118 that falls down along the length of the testicles 200 during intercourse. The protective covering provided by the tube 102 and the shield 118 is effective for inhibiting skin contact between the genitals 206 and testicles 200, and the skin of a sexual partner during intercourse. Consequently, sexually transmitted diseases and pregnancy may be prevented, and the tube 102 is less likely to fall off when the penis 208 softens after semen ejaculation.

As referenced in FIG. 1, the assembly 100 comprises a flexible, thin-walled tube 102 that is sized and dimensioned to substantially cover a penis 208. The tube 102 comprises a closed distal end 104 and an open base end 112. The closed distal end 104 of the tube 102 has a generally smaller diameter than the open base end 112 of the tube 102. In one embodiment, the closed distal end 104 of the tube 102 has a reservoir tip 106 for containing semen.

In another embodiment, the open base end 112 forms a hole 114 for enabling passage of the penis 208. The open base end 112 comprising a perimeter region 116 defined by a circumference 126 that is sized to enable passage of the penis 208 through the hole 114. In yet another embodiment, the tube 102 is defined by an inner surface 108 and an outer surface 110. The inner surface 108 engages the surface of the penis 208.

In one alternative embodiment, the inner surface 108, the outer surface 110, or both of the tube 102 are coated with a lubricant to enhance comfort during intercourse. In some embodiments, the thickness of the tube 102 may be from 0.01 millimeters to 0.25 millimeters. Suitable materials for the tube 102 may include, without limitation, latex, polyurethane, animal skin, and a thin plastic material.

As FIG. 2 illustrates the tube 102 is sized and dimensioned to position over male genitals 206, and specifically the penis 208. The tube 102 has a closed distal end 104 and an open base end 112. The open base end 112 of the tube 102 forms a hole 114 that receives the penis 208. The open base end 112 is defined by a reinforced perimeter region 116 that secures the base end 112 of the tube 102 to the base of the penis 208. In some embodiments, the reinforced perimeter region 116 is reinforced with latex, polyurethane, or plastic. In other embodiments, the perimeter region 116 is defined by a generally circular shape that is sufficiently flexible to adapt to the changing sizes of the penis 208.

In some embodiments, the assembly 100 further comprises a trapezoidal-shaped shield 118. As shown in FIG. 2, the shield 118 acts as an appended barrier to the tube 102, covering the part of the genitals 206 that the tube 102 does not—chiefly the testicles 200. The shield 118 is configured to align and stick to the testicles 200 during intercourse. In this manner, the shield 118 covers a front region 202 and a side region 204 of the testicles 200.

The unique trapezoidal shape of the shield 118 provides sufficient coverage area at shapes and dimensions that align with, and cover the testicles 200 during intercourse. In one non-limiting embodiment, the trapezoidal shape of the shield 118 is generally rectangular. This rectangular length is sufficient to extend longitudinally the entire length of the testicles 200. The trapezoidal shape of the shield 118 may include, without limitation, a right trapezoid, an acute trapezoid, an isosceles trapezoid, a parallelogram, a tangential trapezoid, a Saccheri quadrilateral, a rhombus, and a square. In some embodiments, the size of the shield 118 may be about 25×15 centimeters; the thickness of the shield 118 may be from 0.01 millimeters to 0.25 millimeters. Though in other embodiments, different dimensions may be used.

The shield 118 comprises an attached end 120 and a free end 122. The attached end 120 of the shield 118 is integrally connected with at least half the circumference 126 of the reinforced perimeter of the tube 102—at the base end 112 of the tube 102. In one non-limiting embodiment, the attached end 120 of the shield 118 has a generally shorter length than the free end 122 of the shield 118. In another embodiment, the attached end 120 of the shield 118 forms a pair of obtuse angles (FIG. 1). Thus, the attached end 120 of the shield 118 tapers inwardly to join with the base end 112 of the tube 102. This junction between shield 118 and tube 102 may include welding or other polymer integral connections means known in the art.

In some embodiments, the shield 118 may be fabricated from an adhesive material 124 that helps the shield 118 cling, and thereby wrap around to the testicles 200, and at least partially cling to and cover the scrotum, pubic hair, and peripheral area of the genitals 206. The adhesive material 124 may include, without limitation, a thin polymer film, polyurethane, wax film, a thin latex, a glue, and other clingy materials known in the art.

In use, at first the user inserts the penis 208 into the tube 102 as with a standard condom. The user then rotates the tube 102 until the attached shield 118 is aligned with the testicles 200 (downwardly). The user may then unroll the tube to cover the scrotum, entire pubic hair, and peripheral area. The user may then stretch out the shield 118 to align with the testicles 200. The user may also wrap the shield 118 around the testicles 200 prior to intercourse.

During intercourse, the trapezoidal shape of the shield 118 provides sufficient coverage area to cover the front region 202 and side region 204 of the testicles 200. Also, the adhesive material 124 from which the shield 118 may be fabricated, helps the shield 118 cling to, and thereby wrap around the testicles 200. Also, the adhesive that may coat the shield 118 helps adhere the shield 118 to the testicles 200. In any case, the shield 118 serves as an effective barrier for inhibiting contact between the testicles 200, and the skin of the sexual partner during intercourse.

Although the above description contains many specificities, these should not be construed as limitations on the scope of the invention, but as merely providing illustrations of some of the presently preferred embodiments of this invention. For example, the shield can have other elongated shapes capable of covering the testicles, such as oval shapes, irregular shapes, etc.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A male condom assembly with trapezoidal-shaped shield, the assembly comprising:
a flexible, thin-walled tube comprising a closed distal end and an open base end, the open base end comprising a perimeter region defined by a circumference; and
a trapezoidal-shaped shield comprising an attached end and a free end, the attached end of the shield being integrally connected with at least half the circumference of the reinforced perimeter of the tube.

2. The assembly of claim 1, wherein the shield comprises an adhesive material.

3. The assembly of claim 1, wherein the trapezoidal shape of the shield is generally rectangular.

4. The assembly of claim 1, wherein the trapezoidal shape of the shield includes at least one of the following trapezoidal shapes: a right trapezoid, an acute trapezoid, an isosceles trapezoid, a parallelogram, a tangential trapezoid, a Saccheri quadrilateral, a rhombus, and a square.

5. The assembly of claim 1, wherein the attached end of the shield has a generally shorter length than the free end of the shield.

6. The assembly of claim 1, wherein the attached end of the shield forms a pair of obtuse angles.

7. The assembly of claim 1, wherein the shield has dimensions of about 25×15 centimeters.

8. The assembly of claim 1, wherein the thickness of the shield is from 0.01 millimeters to 0.25 millimeters.

9. The assembly of claim 1, wherein the shield orients downwardly to align with a front region and a side region of the testicles.

10. The assembly of claim 1, wherein the reinforced perimeter region of the tube is reinforced with latex, polyurethane, or plastic.

11. The assembly of claim 1, wherein the perimeter region of the tube is defined by a generally circular shape.

12. The assembly of claim 1, wherein the closed distal end of the tube is defined by a generally smaller diameter than the open base end of the tube.

13. The assembly of claim 1, wherein the closed distal end of the tube comprises a reservoir tip.

14. The assembly of claim 1, wherein the open base end of the tube forms a hole.

15. The assembly of claim 1, wherein the tube comprises a material including at least one of the following: latex, polyurethane, animal skin, and a thin plastic material.

16. The assembly of claim 1, wherein the thickness of the tube is from 0.01 millimeters to 0.25 millimeters.

17. The assembly of claim 1, wherein the tube is defined by an inner surface and an outer surface.

18. The assembly of claim 17, wherein the tube is lubricated from the inner surface, the outer surface, or both to help enhance comfort during intercourse.

19. A male condom assembly with trapezoidal-shaped shield, the assembly comprising:
a flexible, thin-walled tube comprising a closed distal end and an open base end, the open base end comprising a perimeter region defined by a circumference; and
a trapezoidal-shaped shield, the trapezoidal shape of the shield being generally rectangular, the shield comprising an adhesive material, the shield further comprising an attached end and a free end, the attached end of the shield being integrally connected with at least half the circumference of the reinforced perimeter of the tube,
whereby the free end of the shield orients downwardly to align with a front region and a side region of the testicles.

20. A male condom assembly with trapezoidal-shaped shield, the assembly consisting of:
a flexible, thin-walled tube sized and dimensioned to receive a penis, the tube comprising a closed distal end and an open base end, the closed distal end of the tube being defined by a generally smaller diameter than the open base end of the tube, the closed distal end of the tube comprising a reservoir tip, the open base end of the tube comprising a perimeter region defined by a circumference; and
a trapezoidal-shaped shield, the trapezoidal shape of the shield being generally rectangular, the trapezoidal shape of the shield including at least one of the following trapezoidal shapes: a right trapezoid, an acute trapezoid, an isosceles trapezoid, a parallelogram, a tangential trapezoid, a Saccheri quadrilateral, a rhombus, and a square,
the shield comprising an adhesive material, the shield further comprising an attached end and a free end, the attached end of the shield being integrally connected with at least half the circumference of the reinforced perimeter of the tube, the attached end of the shield being defined by a generally shorter length than the free end of the shield, the attached end of the shield forming a pair of obtuse angles,
whereby the free end of the shield orients downwardly align with a front region and a side region of the testicles.
